(19) United States
(12) Patent Application Publication
(10) Pub. No.: US 2024/0316338 A1
(43) Pub. Date: Sep. 26, 2024

(54) ELECTRODE AND GARMENT SYSTEMS AND METHODS FOR IMPROVED DELIVERY OF NEUROMUSCULAR STIMULATION

(71) Applicant: Motive Health, Inc., Scottsdale, AZ (US)

(72) Inventors: Wes Weber, Scottsdale, AZ (US); Kereshmeh Shahriari, Scottsdale, AZ (US); Dharmavirsinh Jhala, Scottsdale, AZ (US)

(21) Appl. No.: 18/611,350

(22) Filed: Mar. 20, 2024

Related U.S. Application Data
(60) Provisional application No. 63/453,361, filed on Mar. 20, 2023.

(51) Int. Cl. A61N 1/04; A61N 1/36

(52) U.S. Cl. CPC A61N 1/0452; A61N 1/0484; A61N 1/0492; A61N 1/3603

(57) ABSTRACT

The disclosure is directed to a system for automatically placing two or more electrodes over different muscle groups. In some embodiments, the system includes an electrode sheet that includes the two or more electrodes. In some embodiments, at least a portion of the two or more electrodes are printed on the electrode sheet, where the printed portion includes a conductive material. In some embodiments, the system includes a garment wrap. In some embodiments, the garment wrap comprises an aperture configured to enable a controller coupling on the electrode sheet to pass through the garment wrap. In some embodiments, the system includes a controller configured to couple to both the garment wrap and the electrode sheet. In some embodiments, the controller is configured to send electrical signals to the two or more electrodes to activate different muscle groups.

### CROSS-REFERENCE TO RELATED APPLICATION

This application claims the priority and benefit of U.S. Provisional Patent Application No. 63/453,361, filed Mar. 20, 2023, which is incorporated by reference herein in its entirety.

### BACKGROUND

Current, typical neuromuscular electrical stimulation (“NMES”) systems require a user to manually place multiple electrodes in specific locations, often resulting in misplacement of electrodes and/or inconsistent placement. Some prior art systems include integrated electrodes into a garment such as a wrap. However, these integrated systems have fixed electrode positioning requiring multiple garments for various portions of the body.

Therefore, there is a need in the art for an NMES system that enables a wide variety of electrode pad configurations while enabling the same garment to be used for multiple portions of the body.

### SUMMARY

In some embodiments, the system is directed to improving an electrically conductive interface between a neuromuscular electrical stimulator (“muscle stimulator”) and a user's skin. In some embodiments, the system includes one or more electrodes each formed on an electrode template configured to properly position one or more electrode pads on a user's body. In some embodiments, the one or more electrodes are non-sterile, intended for single patient use only, and/or are disposable. In some embodiments, electrodes include conductive pads that contact the skin. In some embodiments, the electrical stimulation impulses are delivered through the electrodes on the skin in direct proximity to the muscles to be stimulated. In some embodiments, the electrodes are configured to be worn by a user (e.g., for approximately 30 minutes a day) for multiple uses. In some embodiments, the electrodes configured to be replaced (e.g., every two weeks or 14 uses).

In some embodiments, the electrode (also referred to as an electrode sheet) is configured to be worn on a user's thigh. In some embodiments, the electrode sheet is configured to cover the quadriceps muscles to apply electrical stimulation therapy when connected to a controller which includes a muscle stimulator. In some embodiments, the electrodes include a mirror and/or identical configuration for the right and knee thighs.

In some embodiments, an electrode consists of multiple layers. In some embodiments, a skin-contacting layer of the electrode sheet is comprised of three separate medical grade, self-adhering, biocompatible, and conductive hydrogel (gel) pads. In some embodiments, the gel pads are placed on a single (e.g., polyethylene terephthalate (PET)) sheet. In some embodiments, the gel pads include two rectangular 2 inch×4 inch (5.1×10.2 cm) and one circular, 2.165 inch diameter (5.5 cm) pads, as non-limiting examples. In some embodiments, an overall dimensions of a non-limiting example electrode sheet are 15.6×21.8 cm (6.14×8.58 in). In some embodiments, the pads can comprise any desired shape.

In some embodiments, the outermost layer of the electrode includes a magnetic polygonal (e.g., triangle) shaped connector housing for electrical connection to the muscle stimulator. In some embodiments, the electrode includes a flex circuit with printed silver ink electrical traces on the (PET) film that is configured to provide electrical conductivity from the muscle stimulator to the gel pads. In some embodiments, the silver traces are covered by a polypropylene dielectric layer protecting the traces. In some embodiments, when not in use, the electrode sheet is covered by a (paper-backed) release liner.

In some embodiments, the disclosure is directed to a system for positioning electrodes on muscle groups. In some embodiments, the system comprises one or more of an electrode sheet, a flexible circuit, and two or more electrodes. In some embodiments, the flexible circuit is coupled to the electrode sheet. In some embodiments, the two or more electrodes are coupled to the electrode sheet. In some embodiments, the two or more electrodes are positioned on the electrode sheet to each activate a different muscle group.

In some embodiments, the electrode sheet comprises an adhesive. In some embodiments, the adhesive is configured to hold the electrode sheet in fixed position on a user. In some embodiments, the adhesive is located between the two or more electrodes. In some embodiments, the electrode sheet is configured to not wrap completely around a user limb.

In some embodiments, the electrode sheet comprises non-conductive portion. In some embodiments, at least a portion of each of the two or more electrodes on the electrode sheet comprise a conductive material printed on the non-conductive portion.

In some embodiments, the electrode sheet comprises a controller coupler. In some embodiments, the controller coupler is positioned on an opposite side of the electrode sheet than the two or more electrodes. In some embodiments, the controller coupler comprises a raised portion configured to guide controller inputs to controller contacts on the electrode sheet.

In some embodiments, the system further includes a flexible garment. In some embodiments, the flexible garment is configured to wrap around a user's limb. In some embodiments, the flexible garment is configured to secure to itself when wrapped around the user's limb. In some embodiments, the flexible garment comprises one or more of a main body portion, a left extending arm, and a right extending arm. In some embodiments, the main body portion comprises a controller attachment portion comprising an aperture. In some embodiments, the aperture is configured to enable the raised portion to pass through the aperture.

In some embodiments, the controller is configured to couple to the controller attachment portion. In some embodiments, the controller is held in position by the raised portion. In some embodiments, the controller coupler, the aperture, and/or the controller attachment portion all comprise substantially a same shape.

In some embodiments, the controller is configured to deliver electricity to the two or more electrodes. In some embodiments, the electrode sheet includes a thigh sheet. In some embodiments, each thigh sheet includes an electrode placement for two or more muscle groups from muscle groups comprising a Rectus Femoris muscle group, a Vastus Lateralis muscle group, and/or a Vastus Medialis muscle group.

In some embodiments, each of the two or more electrodes include a conductive material. In some embodiments, the conductive material includes a grid pattern. In some embodiments, the grid pattern is printed onto the electrode sheet.

### DRAWING DESCRIPTION

FIG. 1 shows a non-limiting assembled view of the system according to some embodiments.

FIG. 2 illustrates the placement of the electrode on a patient's thigh before a wrap is applied according to some embodiment.

FIG. 3 depicts the application of a wrap over the electrode according to some embodiments.

FIG. 4 illustrates a non-limiting example of how a user can wear the system according to some embodiments.

FIG. 5 shows a view of a skin contact side of the electrode according to some embodiments.

FIG. 6 shows an electrode top side including a triangle shaped coupling and magnetic connectors according to some embodiments.

FIG. 7 shows a skin contact side cover according to some embodiment.

FIG. 8 shows a controller top according to some embodiment.

FIG. 9 shows a controller bottom according to some embodiments.

FIG. 10 illustrates another view of an electrode top side according to some embodiments.

FIG. 11 shows another view of the skin contact side according to some embodiments.

FIG. 12 shows various components of a right electrode according to some embodiments.

FIG. 13 shows various components of a left electrode according to some embodiments.

FIG. 14 illustrates how the electrode pads for a left knee electrode are arranged for treatment of left knee pain according to some embodiments.

FIG. 15 shows a first electrode pattern according to some embodiments.

FIG. 16 shows a second electrode pattern according to some embodiments.

FIG. 17 shows a third electrode pattern according to some embodiments.

FIG. 18 shows a fourth electrode pattern according to some embodiments.

FIG. 19 shows a fifth electrode pattern according to some embodiments.

FIG. 20 illustrates the multiple layer configuration of a right leg electrode according to some embodiments.

FIG. 21 shows further details of the circuit and pattern arrangement for a right thigh electrode according to some embodiments.

FIG. 22 illustrates additional details of the circuit pattern assembled on an electrode template according to some embodiments.

FIG. 23 illustrates how the matching shaped coupling ensures proper positioning of the wrap over the electrode according to some embodiments.

FIG. 24 depicts coupling the controller to the electrode and/or garment according to some embodiments.

FIG. 25 illustrates internal components of the controller and the electrical interface with the conductive pattern according to some embodiments.

FIG. 26 shows an exploded view of an electrode according to some embodiments.

FIG. 27 shows a system QR code and system graphical user interfaces according to some embodiments.

FIG. 28 shows impedance test data according to some embodiments.

FIG. 29 illustrates a computer system 110 enabling or comprising the systems and methods in accordance with some embodiments.

### DETAILED DESCRIPTION

In some embodiments, systems and methods described herein (collectively referred to as the “system”) are directed to a therapeutic medical device configured to apply a novel neuromuscular electrical stimulation (NMES) therapy to strengthen and/or rehabilitate muscles and/or joints and/or to treat joint pain. In some embodiments, the joint pain is associated with arthritis. In some embodiments, the system includes one or more controllers, pulse generators, garments, and/or electrodes. In some embodiments, a garment includes a wrap. As used herein, any reference to a specific species (e.g., wrap, arthritis) is also a reference to, and interchangeable with, the genus (e.g., garment, joint pain) when defining the metes and bounds of the system.

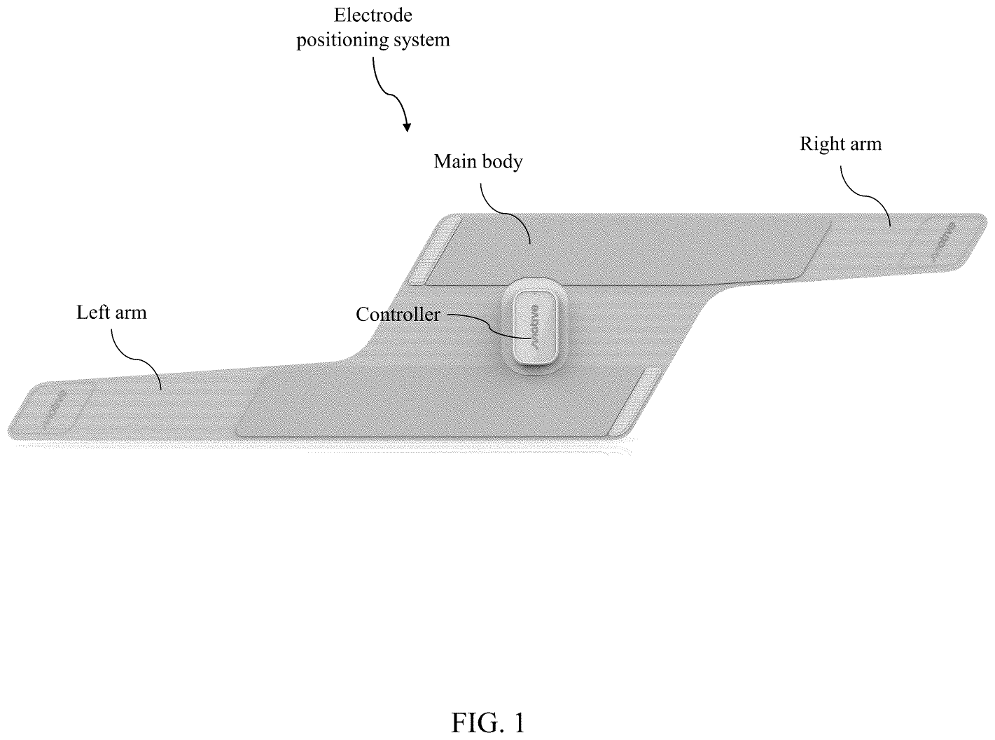

FIG. 1 shows a non-limiting assembled view of the system according to some embodiments. In some embodiments, the garment is configured to provide support and/or protection to an electrode positioned over a portion of a user's body (e.g., the thigh) to deliver NMES therapy to one or more muscles (e.g., the quadriceps muscles). In some embodiments, the system includes a pulse generator and/or controller (collectively referred to herein as a “controller”) configured to be connected to the electrode using a matching shaped coupling interface. In some embodiments, the matching shape coupling interface includes a triangle block interface. In some embodiments, the system is configured to enable the wrap to be placed over an electrode and under the controller to secure the electrode sheet on the skin and provide compression.

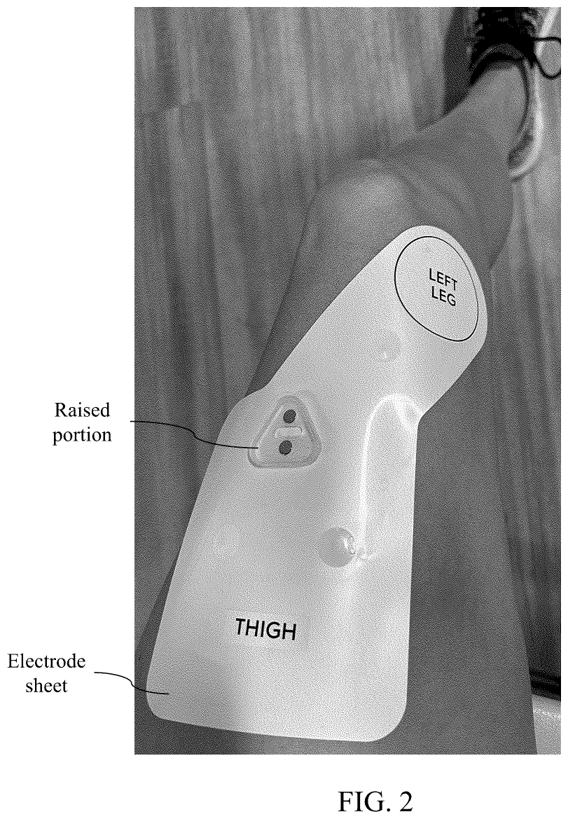

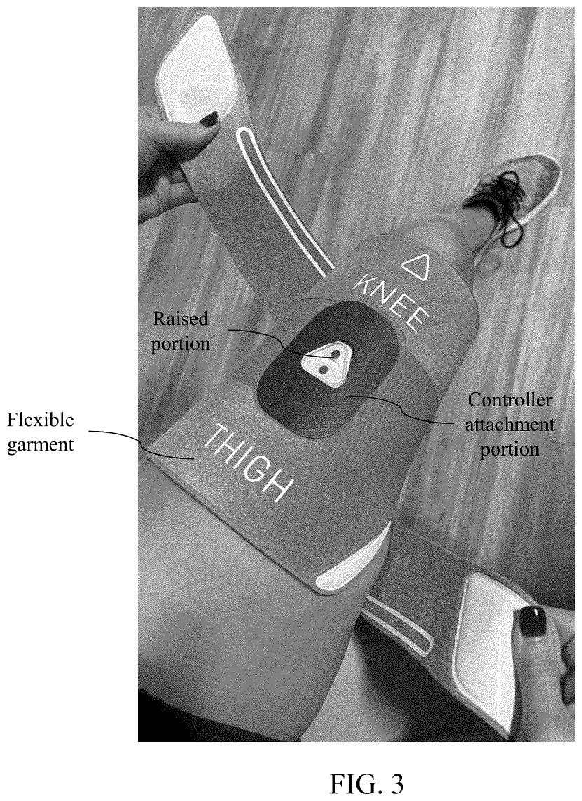

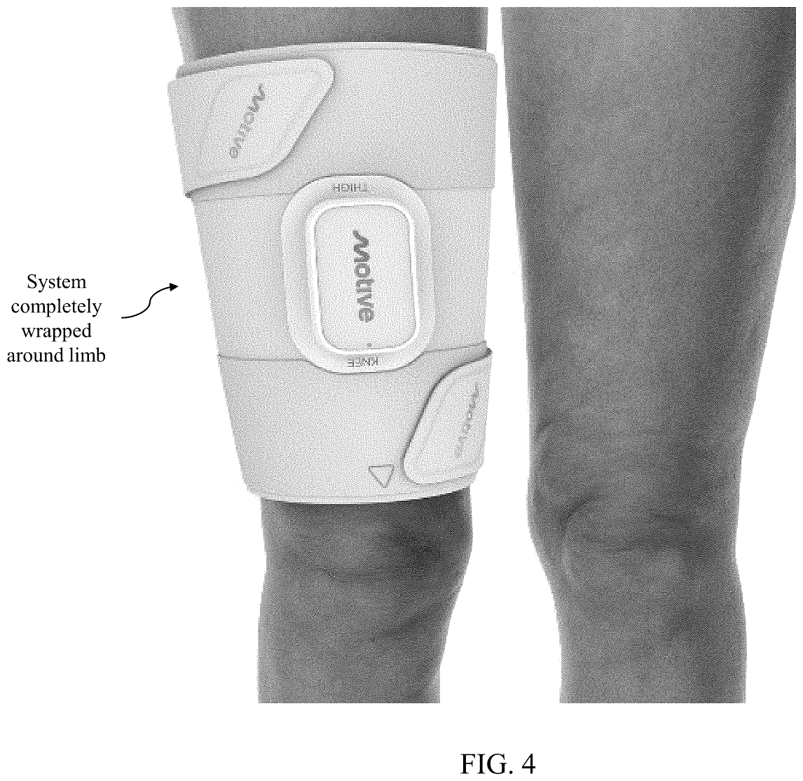

FIG. 1 shows a non-limiting assembled view of the system according to some embodiments. FIG. 2 illustrates the placement of the electrode on a patient's thigh before a wrap is applied according to some embodiment. FIG. 3 depicts the application of a wrap over the electrode according to some embodiments. FIG. 4 illustrates a non-limiting example of how to wear the system according to some embodiments.

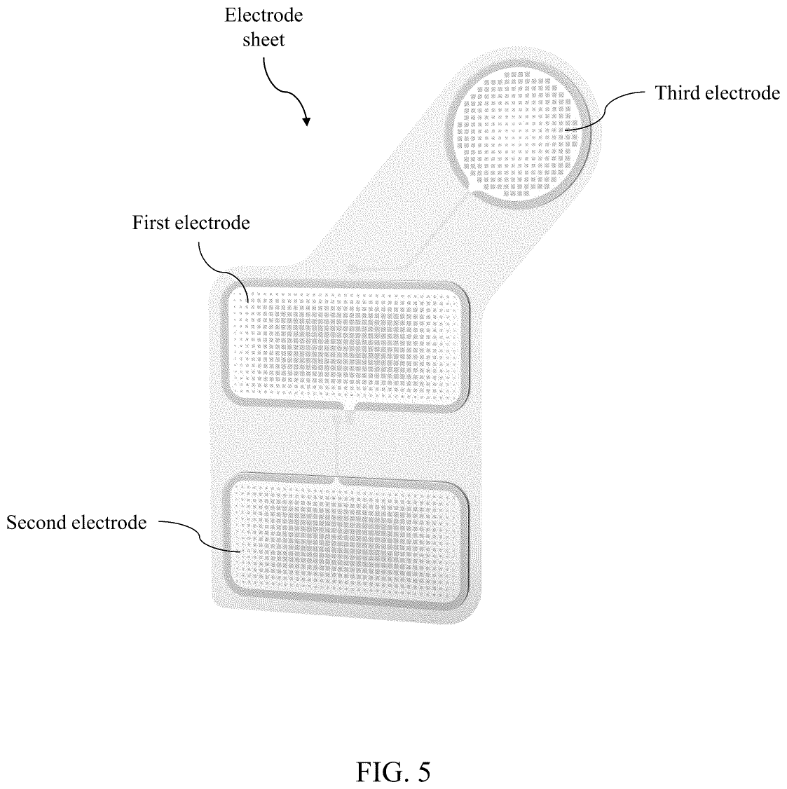

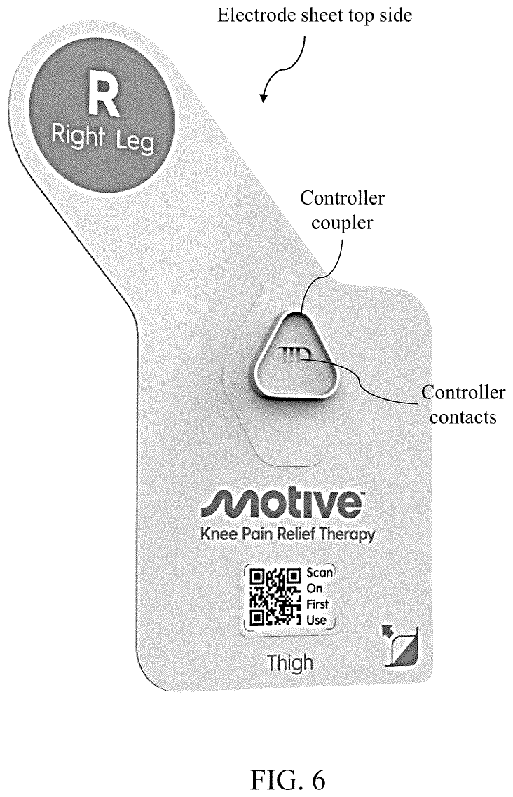

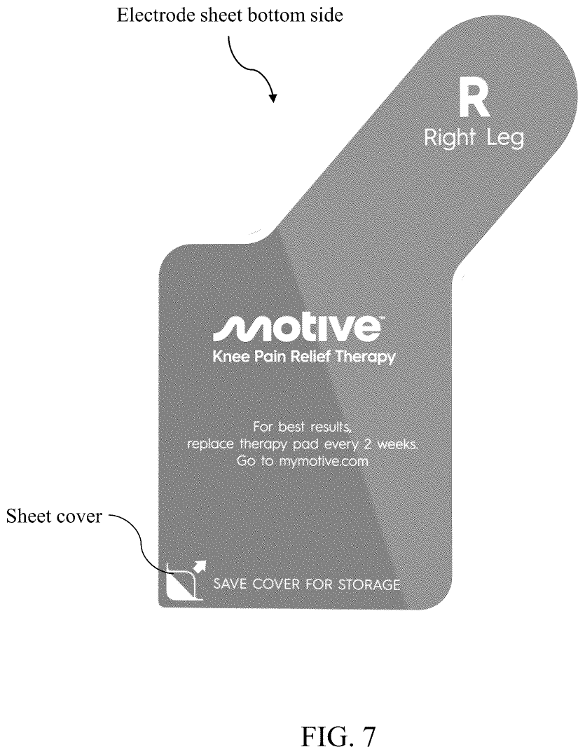

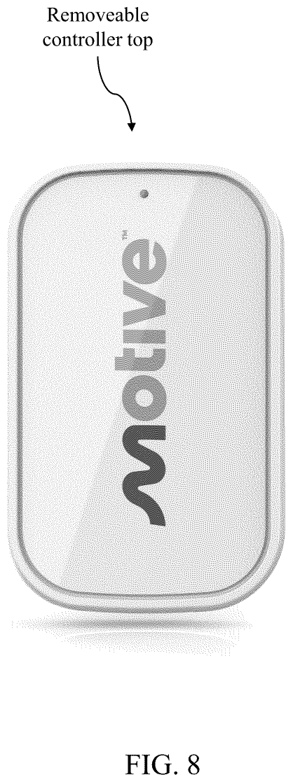

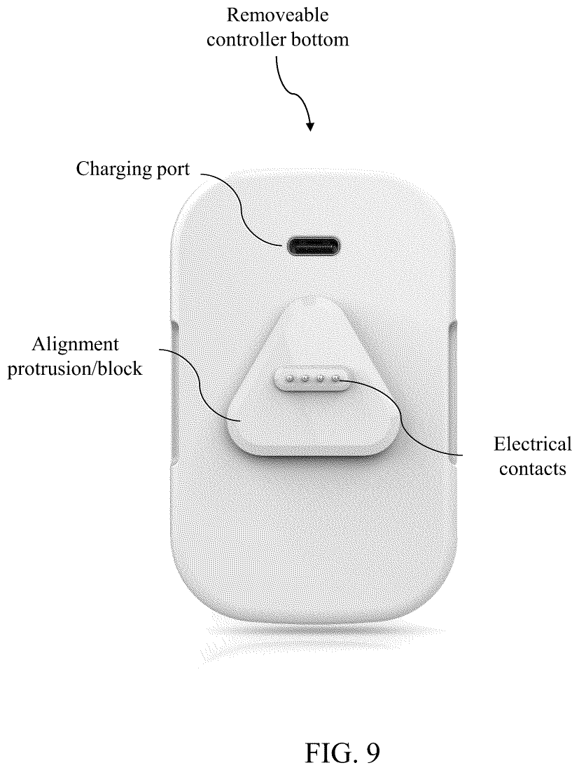

FIG. 5 shows a view of a skin contact side of the electrode according to some embodiments. In some embodiments, the skin contact side includes one or more electrically conductive pads which include a conductive gel. FIG. 6 shows an electrode top side including a triangle coupling and magnetic connectors according to some embodiments. FIG. 7 shows a skin contact side cover according to some embodiment. In some embodiments, the skin contact cover is configured to protect electrodes from drying. FIG. 8 shows a controller top according to some embodiment. FIG. 9 shows a controller bottom according to some embodiments. In some embodiments, the controller bottom comprises a polygonal (e.g., triangular) and/or magnetic connector.

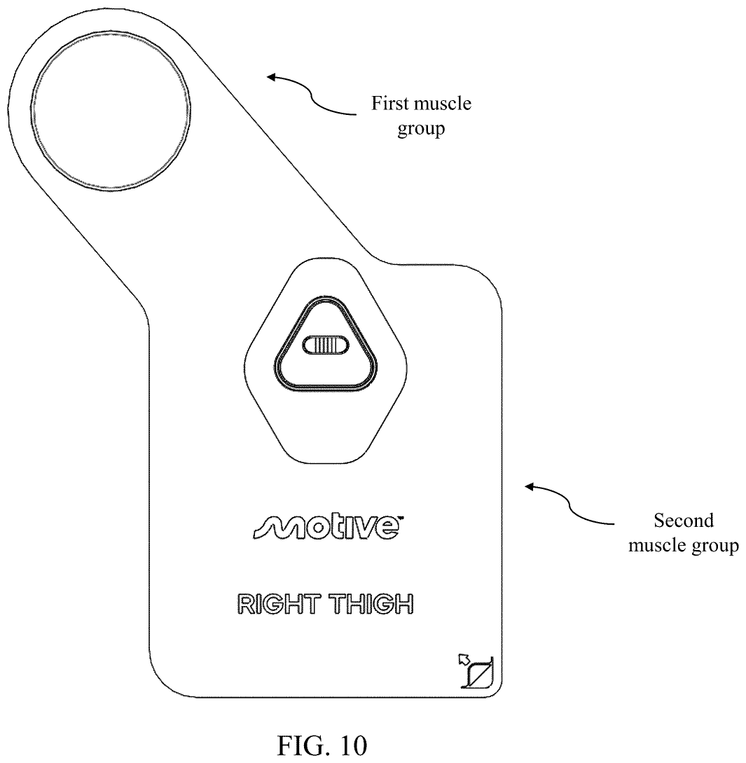

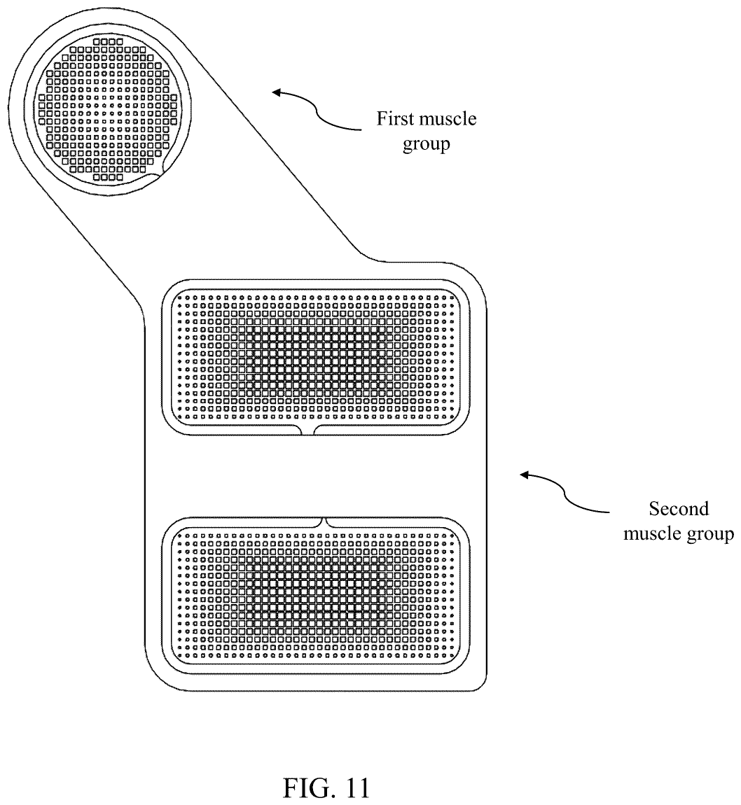

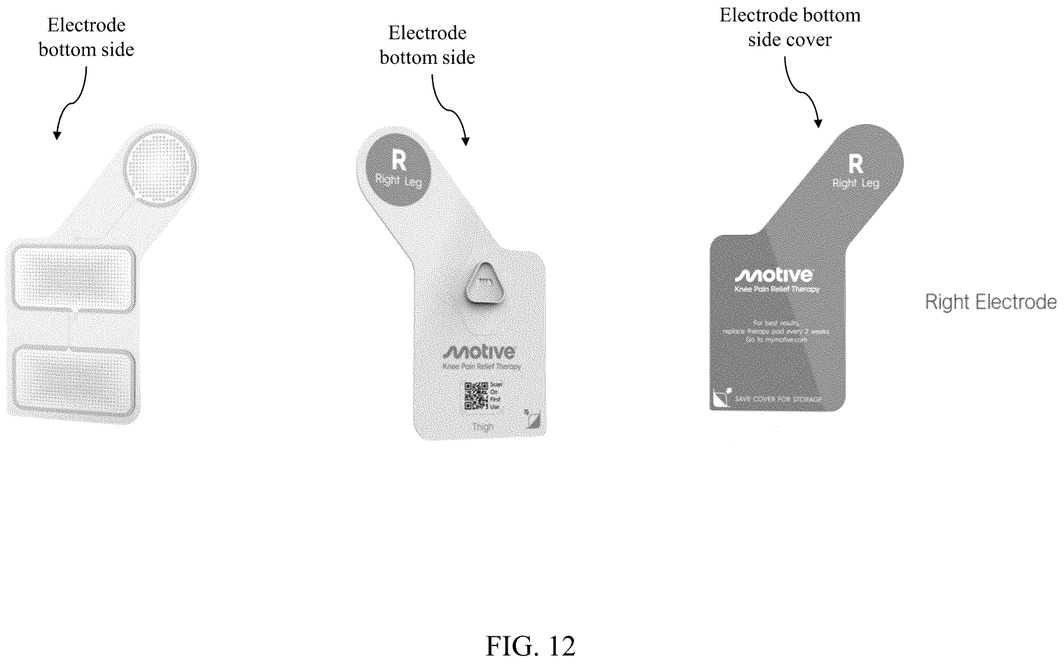

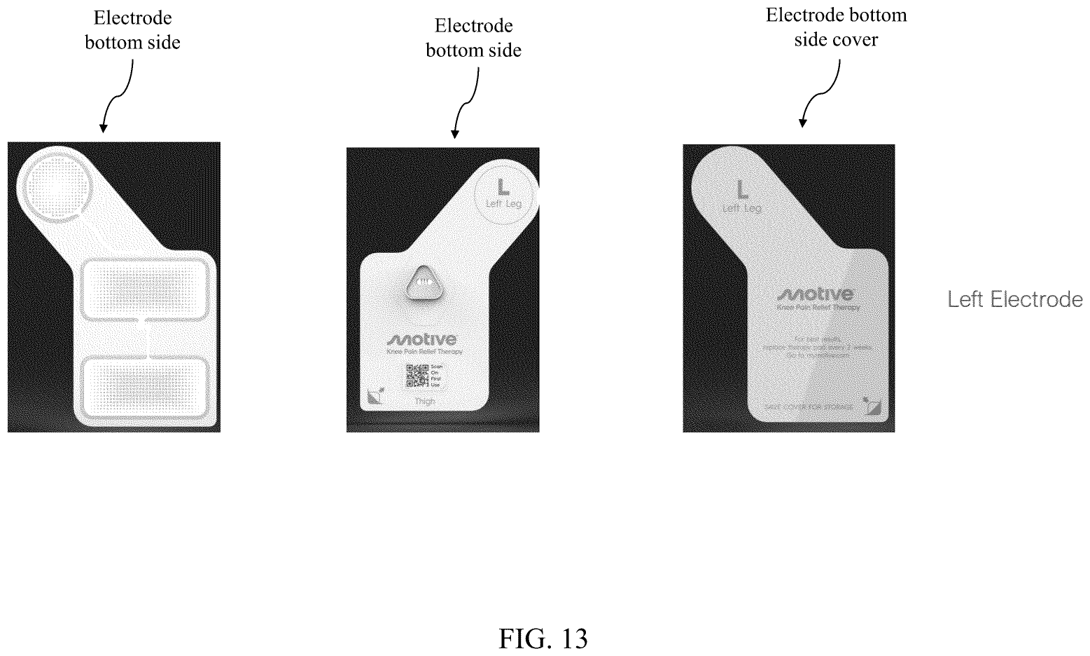

FIG. 10 illustrates another view of an electrode top side according to some embodiments. In some embodiments, one or more electrodes are configured and/or shaped for application on a specific area of the body. FIG. 11 shows another view of the skin contact side according to some embodiments. In some embodiments, one or more electrodes comprise a varying diameter and/or varying shape pattern configured to distribute electrical current in a pre-determined fashion. FIG. 12 shows various components of a right electrode according to some embodiments. FIG. 13 shows various components of a left electrode according to some embodiments.

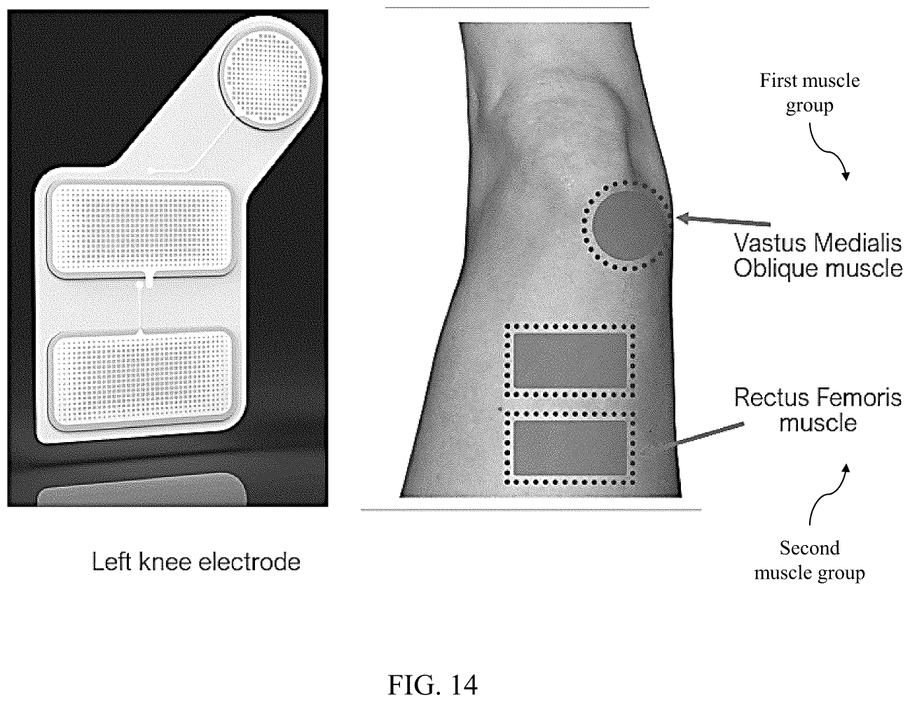

FIG. 14 illustrates how the electrode pads for a left knee electrode are arranged for treatment of left knee pain according to some embodiments. In some embodiments, each electrode described herein is configured for treatment of a specific area of the body. In some embodiments, one or more electrode pads pre-arranged on an electrode template which provides the benefit of consistent placement and/or stimulation for maximum effectiveness. For example, according to the non-limiting arrangement shown in FIG. 14, a left knee electrode is configured to apply pain therapies by ensuring a proper covering of the vastus medialis oblique (VMO) and rectus femoris (RF) muscles. In some embodiments, these muscles of quadriceps play a significant role in off-loading of the knee joint. In some embodiments, when these two muscles are activated and strengthened over time by application of NMES therapy, they reduce the compressive loads from the knee joint resulting in knee pain relief and improving mobility. However, conventional systems often lead to patients misplacing electrodes which produces an variety of undesirable results. Therefore, in this non-limiting example, the electrode pad is pre-arranged to cover the VMO and RF muscle on the right thigh or left thigh.

In some embodiments, one or more sheets are configured, arranged, and/or positioned to activate muscle groups within close proximity. In some embodiments, a sheet is configured to cover two or more muscle groups. In some embodiments, a sheet includes two or more electrodes each positioned to overlay a separate muscle group. In some embodiments, the muscle group proximity is between 4 and 12 inches. In some embodiments, the muscle group proximity is between 6 to 10 inches.

In some embodiments, one or more sheets include one or more shoulder sheets. In some embodiments, a shoulder sheet includes an electrode placement for one or more of Deltoid, Infraspinatus, and/or Teres Major muscle groups. In some embodiments, one or more sheets include one or more thigh sheets. In some embodiments, a thigh sheet includes an electrode placement for one or more of Rectus Femoris, Vastus Lateralis, and/or Vastus Medialis muscle groups. In some embodiments, one or more sheets include one or more ankle sheets. In some embodiments, an ankle sheet includes electrode placement for one or more of Gastrocnemius, Soleus, and Tibialis Anterior muscle groups. In some embodiments, one or more sheets include a forearm sheet. In some embodiments, a forearm sheet includes electrode placement for one or more of Flexor Carpi Radialis, Palmaris Longus, and Flexor Carpi Ulnaris muscle groups. In some embodiments, one or more sheets include a back sheet. In some embodiments, a back sheet includes electrode placement for one or more of Erector Spinae, Latissimus Dorsi, and Trapezius muscle groups.

In some embodiments, one or more electrodes includes an electrode pattern configured to uniformly distribute electrical current. In some embodiments, one or more electrodes include a specific pattern for a specific joint and/or area of the body. In some embodiments, one or more electrode patterns include a conductive material. In some embodiments, the conductive material includes a grid patterned conductive material. In some embodiments, the first electrode pattern includes a lower conductivity area in the center, and a higher conductivity area around the parameter. In some embodiments, a lower conductivity area includes less conductive material than a higher conductivity area. In some embodiments, a lower conductivity area includes holes in-between conductive material that are larger than a higher conductivity area. In some embodiments, the center holes includes holes are within 3 holes of the center of the electrode pattern. In some embodiments, parameter holes include holes that are within 3 holes of the pattern edge.

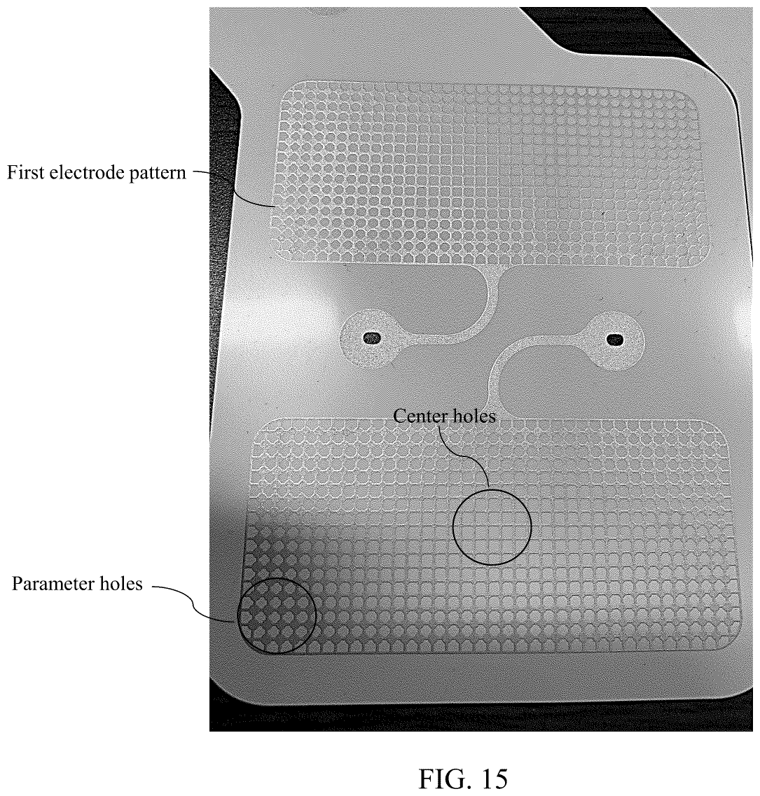

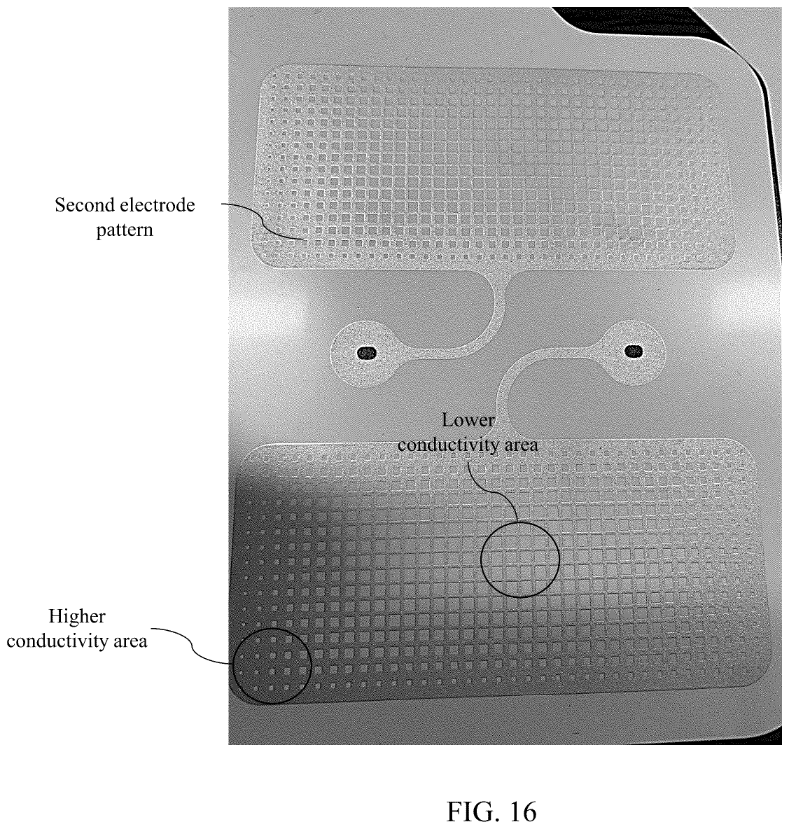

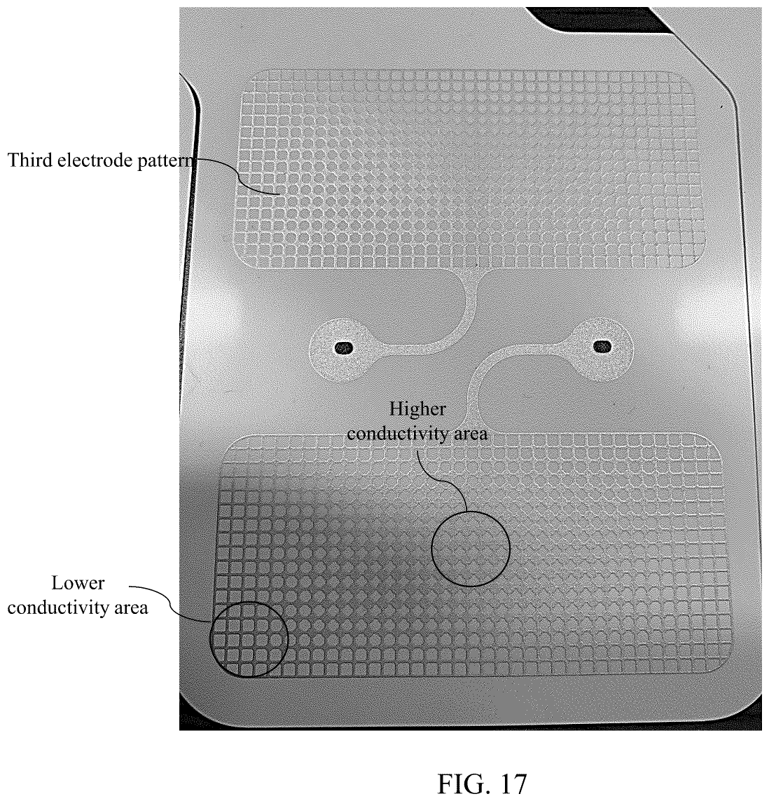

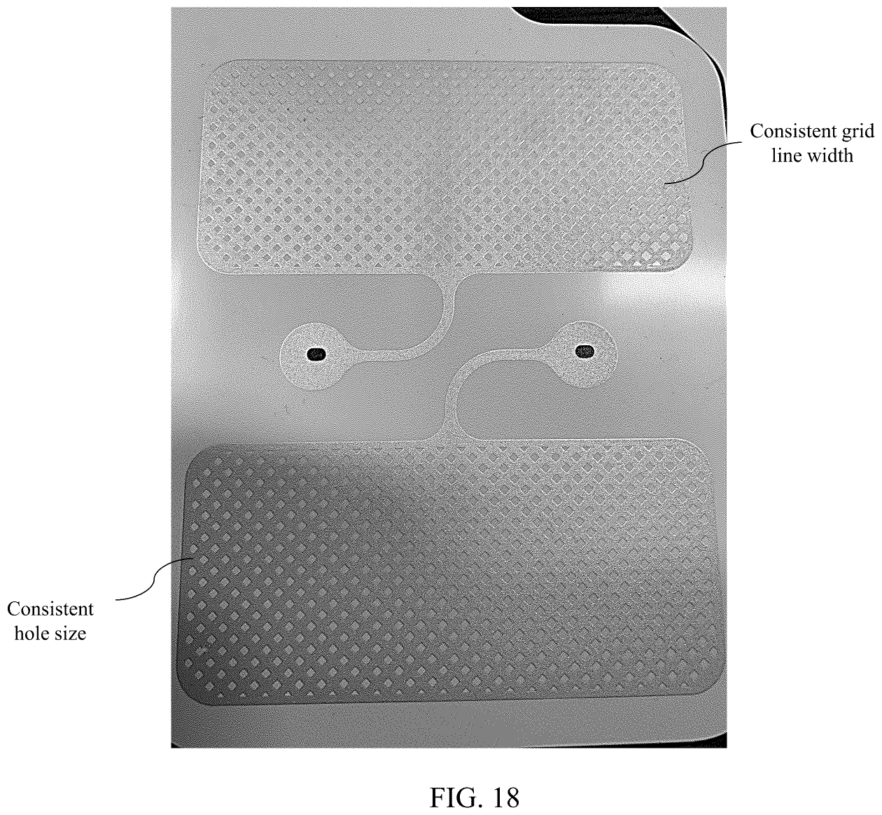

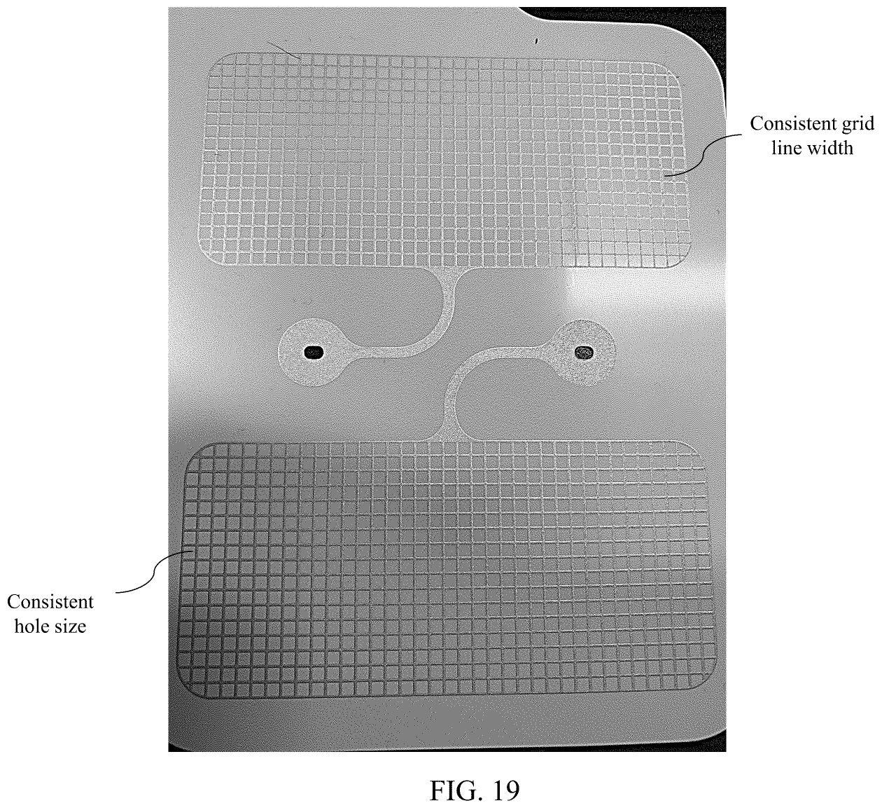

FIG. 15 shows a first electrode pattern according to some embodiments. In some embodiments, the first electrode pattern parameter includes parameter holes that are 60-90% of the area of the center holes. FIG. 16 shows a second electrode pattern according to some embodiments. In some embodiments, the second electrode pattern parameter includes parameter holes that are 30-70% of the area of the center holes. FIG. 17 shows a third electrode pattern according to some embodiments. In some embodiments, the third electrode pattern parameter includes center holes that are 10-50% of the area of the parameter holes. FIG. 18 shows a fourth electrode pattern according to some embodiments. In some embodiments, the fourth electrode pattern includes a grid pattern with a hole size variation less than 10% across the electrode pattern. In some embodiments, the fourth electrode pattern hole density is between 20-50% of the electrode pattern. FIG. 19 shows a fifth electrode pattern according to some embodiments. In some embodiments, the fifth electrode pattern hole density is between 50-80% of the electrode pattern. In some embodiments, the electrode patterns vary in density in different directions for improved therapeutic effect.

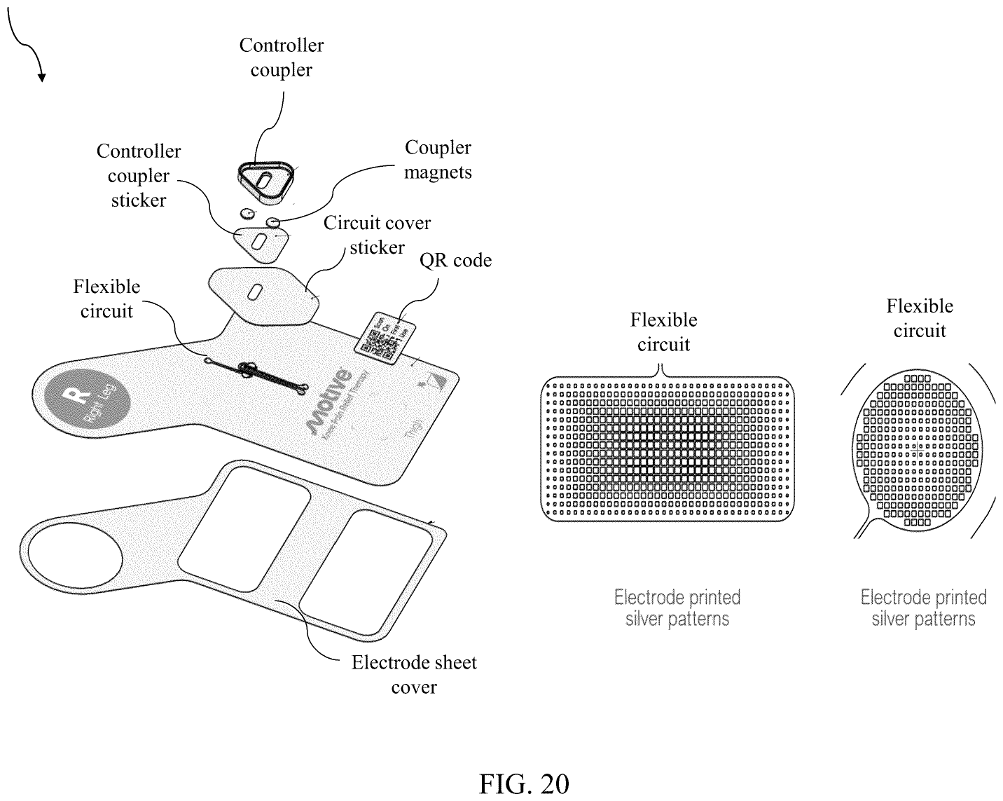

FIG. 20 illustrates the multiple layer configuration of a right leg electrode according to some embodiments. In some embodiments, each electrode sheet is configured for a specific joint and comprises a multiple layer configuration. In some embodiments, the multiple layer configuration is similar to that shown in FIG. 20 with the exception of the arrangement of the electrode template in one or more electrodes.

In some embodiments, layer 1 includes a mechanical connection to a stimulator. In some embodiments, layer 1 is keyed to a single connection orientation with magnetically generated downward force ensuring a robust electrical connection to the stimulator pogo pins (see FIGS. 15-19).

In some embodiments, layer 2 includes a shaped dielectric layer protecting the conductive traces. FIG. 17 shows a diamond shaped dielectric layer according to some embodiments.

In some embodiments, layer 3 includes conductive (e.g., silver ink) traces configured to electrically interface to the stimulator and pass the electrical connection to the bottom side of the electrode sheet through vias (see FIGS. 15-19).

In some embodiments, layer 4 includes a template sheet that is semi-pliable (e.g., polyethylene terephthalate (“PET”) to conform to a body part (e.g., a thigh) and/or serves as the hydrogel carrier substrate and top to bottom trace dielectric (see FIG. 15).

In some embodiments, layer 5 includes one or more (e.g., three) electrode pattern grids comprising conductive (e.g., silver) ink traces that electrically distribute the stimulator's electrical energy output to the three conductive hydrogel pads placed on the grids of electrodes. In some embodiments, two grids are provided in a rectangular pattern covering the top part of thigh muscle (rectus femoris muscle) and a third one is provided in a circular form covering the vastus medialis oblique muscle (See FIGS. 15-19). In some embodiments, the gel pads are 2 inch×4 inch (5.1×10.2 cm) and, 2.165 inch diameter (5.5 cm) pad, as non-limiting examples.

In some embodiments, layer 6 includes a dielectric layer protecting the grid formed from silver ink traces from skin contact. In some embodiments, the dielectric layer correlates to substantially the dimensions of conductive hydrogel electrodes.

In some embodiments, layer 7 includes a medical grade, self-adhering, biocompatible conductive hydrogel that covers the electrode pattern grids silver ink, which results in an electrical connection to the skin contacting the electrodes and/or gel. In some embodiments, a non-limiting example hydrogel type is KM-40C manufactured by Katecho, Inc., Des Moines, IA.

In some embodiments, a method of manufacture and/or a product by process includes building one or more layers described herein.

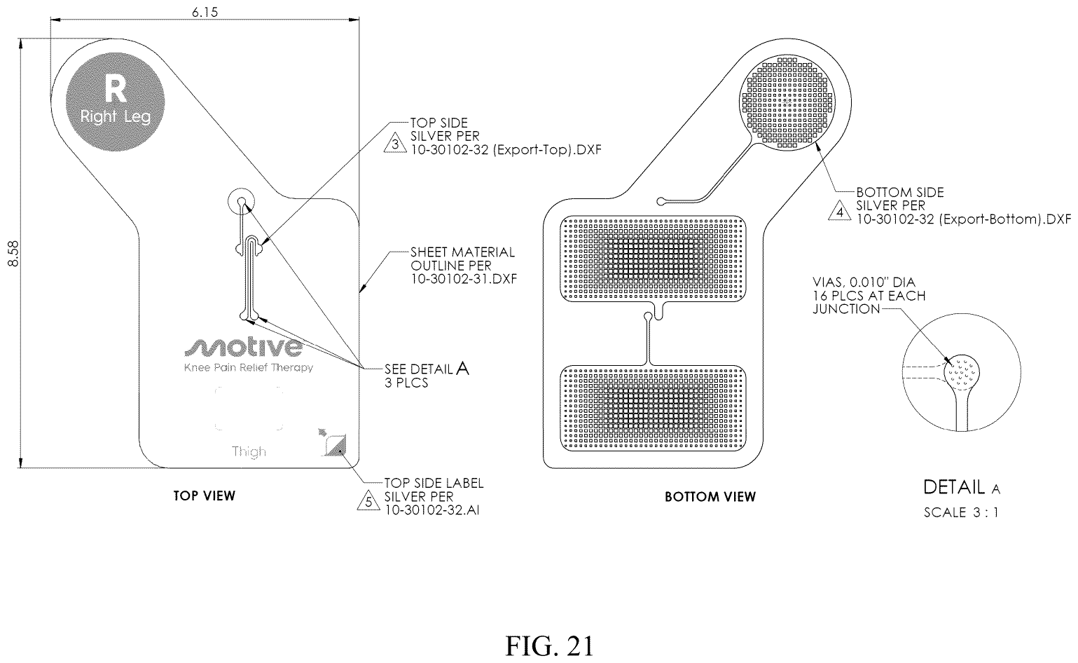

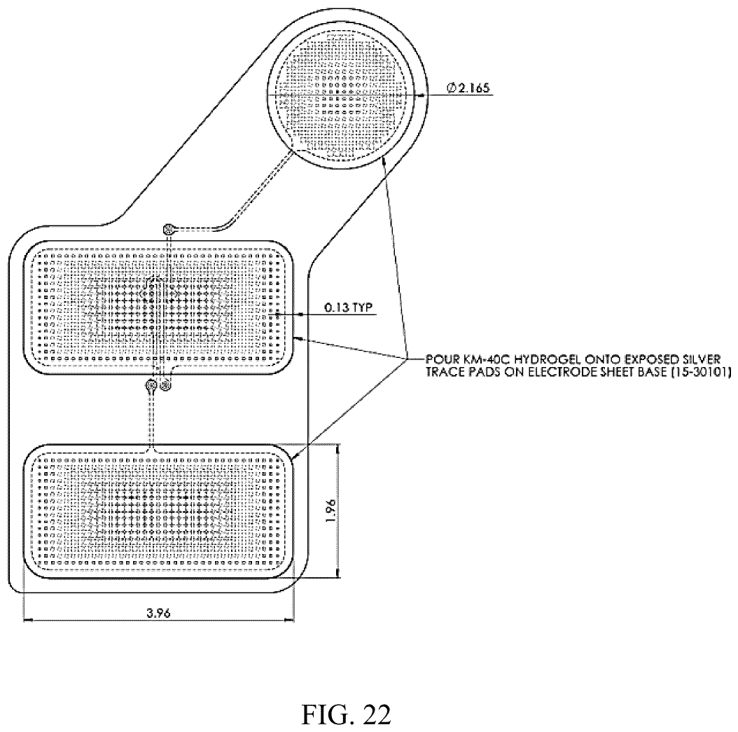

FIG. 21 shows further details of the circuit and pattern arrangement for a right thigh electrode according to some embodiments. FIG. 22 illustrates additional details of the flex circuit pattern assembled on an electrode template according to some embodiments. In some embodiments, a method of manufacture includes poring hydrogel into exposed trace pads on the electrode template. In some embodiments, the system includes printed silver traces configured to transfer electrical current from the controller to the electrode printed silver grids.

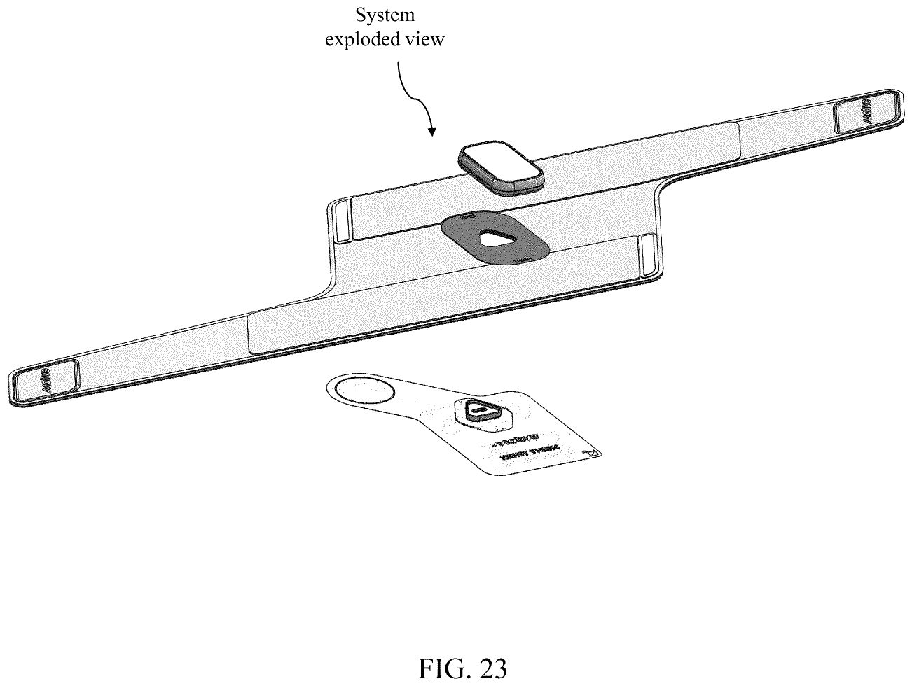

FIG. 23 shows the electrode connection being placed through the garment and connected to the controller according to some embodiments. In some embodiments, the non-limiting example “triangle” interface eliminates any potential for error by the user placing the controller on the electrode in a wrong orientation as the triangle in the bottom of controller must match the triangle on the electrode sheet. Other interface shapes and sizes may be used in some embodiments.

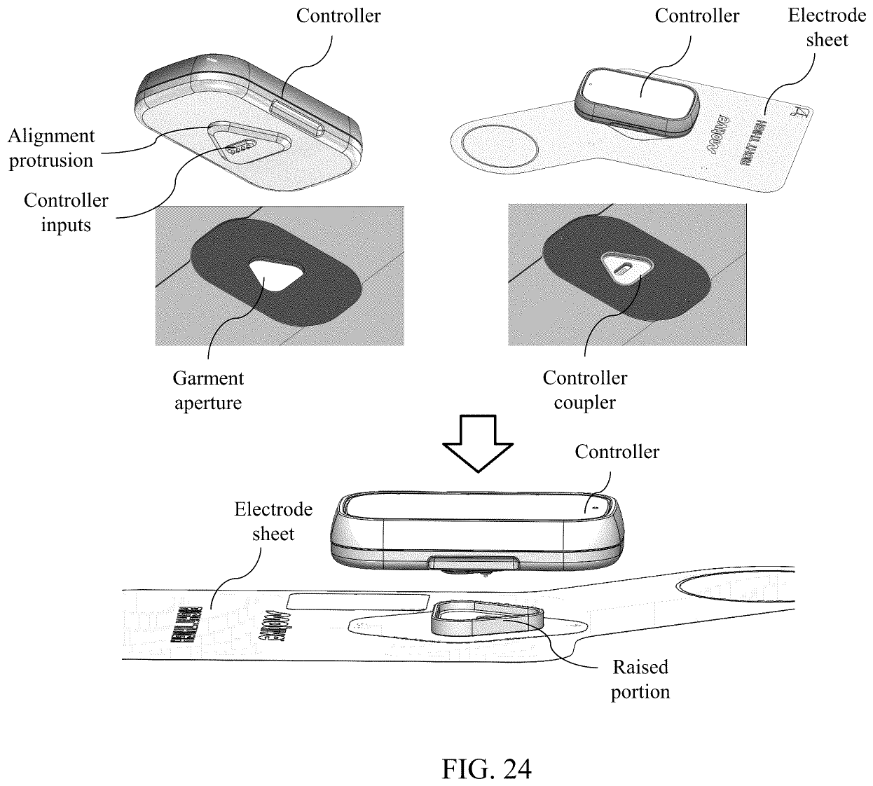

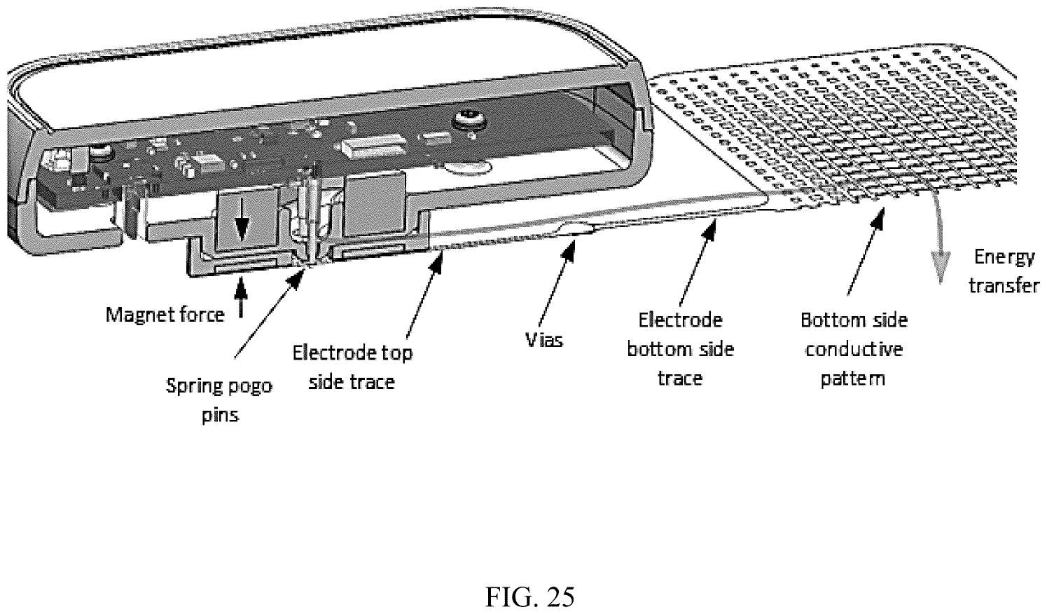

In some embodiments, the controller is coupled to and/or seated on the electrode sheet using a magnetic interface. In some embodiments, the magnetic force secures the attachment and eliminates a “loose” connection between the two parts. FIG. 24 depicts coupling the controller to the electrode according to some embodiments. FIG. 25 illustrates internal components of the controller and the electrical interface with the conductive pattern according to some embodiments.

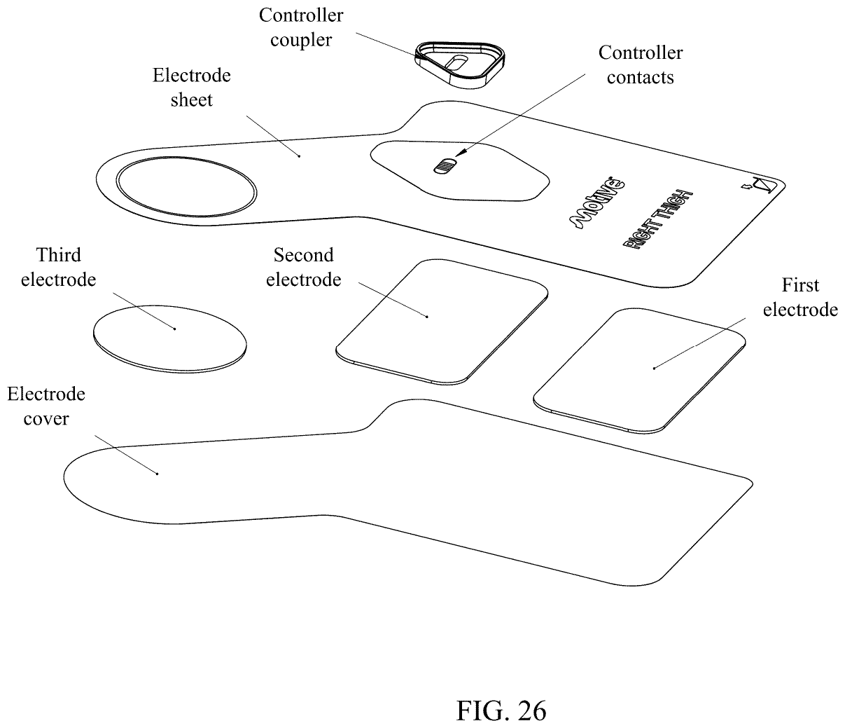

FIG. 26 shows an exploded view of an electrode according to some embodiments. In some embodiments, a method of manufacture includes arranging the one or more components as shown in this non-limiting example. In some embodiments, the system includes a kit comprising one or more separated multi-layer electrodes, garments, and/or controllers manufactured and/or arranged according to some embodiments.

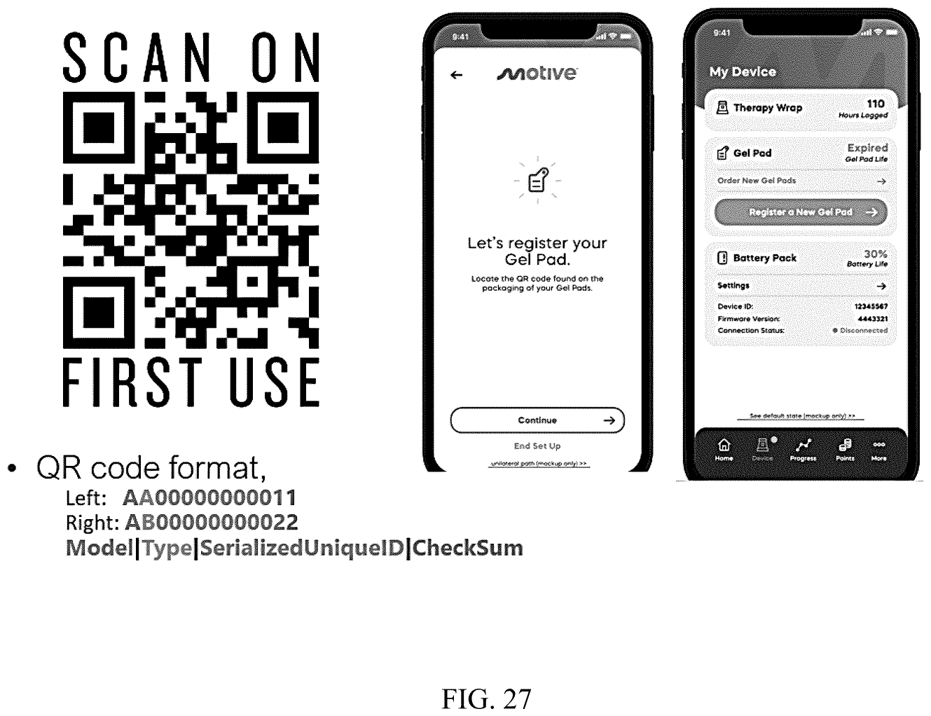

FIG. 27 shows a system QR code and system graphical user interfaces (“GUIs”) according to some embodiments. In some embodiments, a GUI is configured to display the electrode usage. In some embodiments, the GUI includes part of an application (“App”) executing one or more program instructions stored on one or more non-tangible computer readable media. In some embodiments, the GUI is configured to be generated on any type of display such as those described herein.

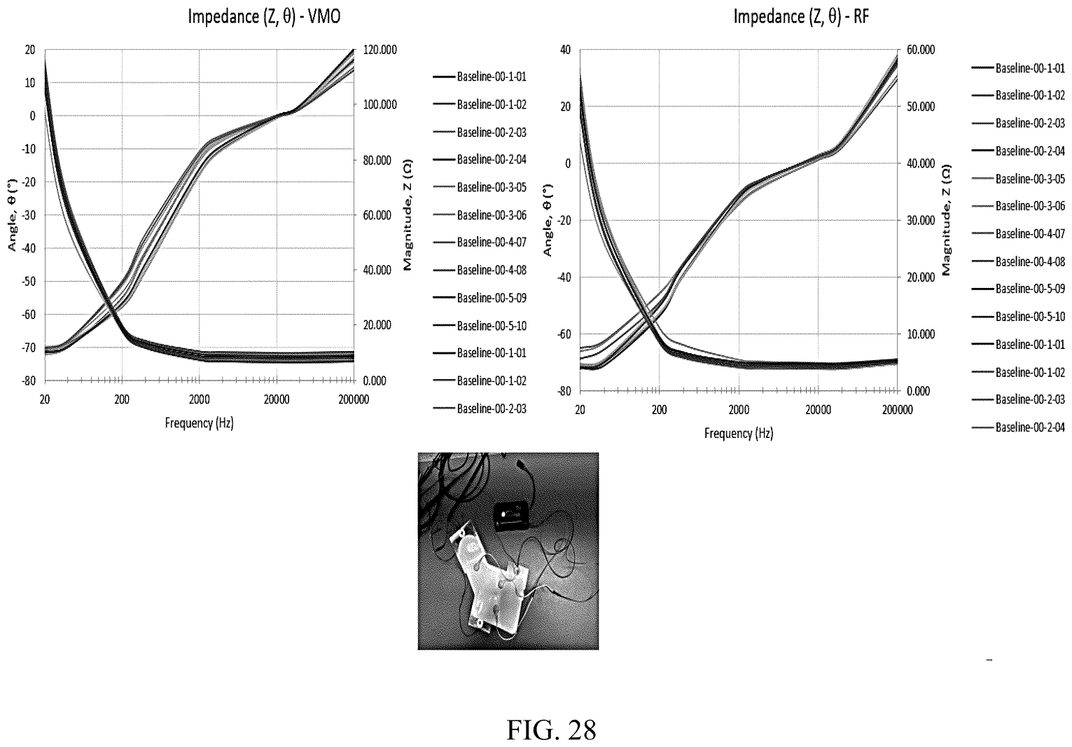

In some embodiments, the system is configured to scan and/or track electrode usage. In some embodiments, the system is configured to predict and/or alert a user when an electrode should be replaced and/or remaining electrode life. In some embodiments, one or more electrodes are consumable item with a limited life (e.g., 14 therapy sessions). In some embodiments, time of use, delays between uses and/or treatment intensity are tracked to provide electrode life input. Remaining electrode life can communicated to the user using alarms or other notifications in some embodiments. In some embodiments, by alerting users, safety is improved by minimizing the risk of skin burn, as a non-limiting example. In some embodiments, an electrode includes a QR code placed on and/or integral to an electrode sheet. The QR code can be scanned and provides initiation for the electrode life function. Near field or other wireless communication and/or passive or active radiofrequency identification can be used for initiating and/or tracking electrode life. FIG. 28 shows impedance test data according to some embodiments.

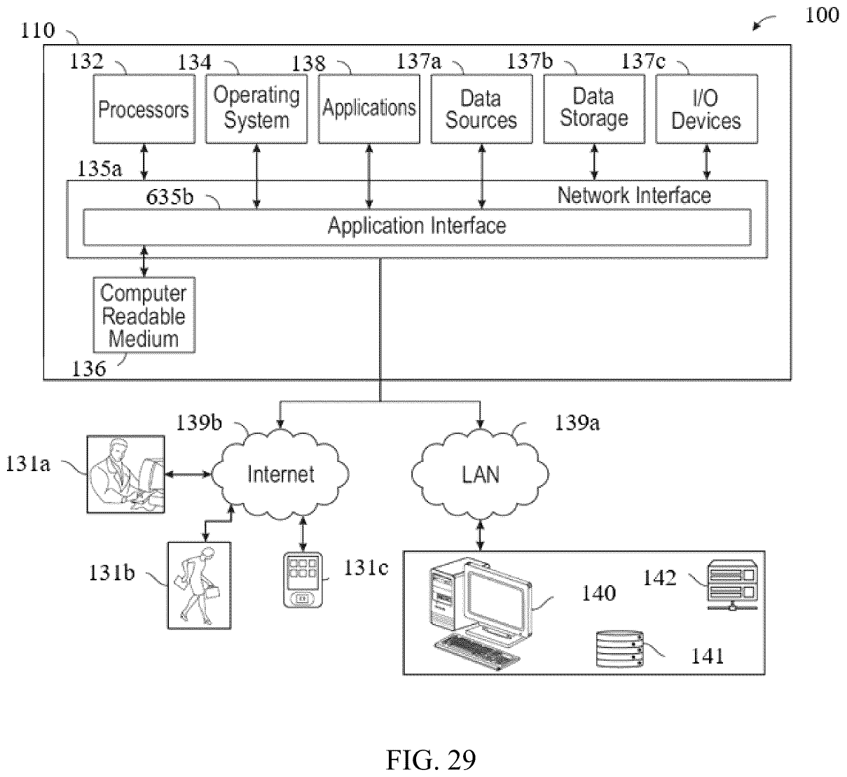

FIG. 29 illustrates a computer system 110 enabling or comprising the systems and methods in accordance with some embodiments. In some embodiments, the computer system 110 is configured to operate and/or process computer-executable code of one or more software modules of the aforementioned system and method. Further, in some embodiments, the computer system 110 is configured to operate and/or display information within one or more graphical user interfaces (e.g., HMIs) integrated with or coupled to the system.

In some embodiments, the computer system 110 comprises one or more processors 132. In some embodiments, at least one processor 132 resides in, or is coupled to, one or more servers. In some embodiments, the computer system 110 includes a network interface 135a and an application interface 135b coupled to the least one processor 132 capable of processing at least one operating system 134. Further, in some embodiments, the interfaces 135a, 135b coupled to at least one processor 132 are configured to process one or more of the software modules (e.g., such as enterprise applications 138). In some embodiments, the software application modules 138 includes server-based software. In some embodiments, the software application modules 138 are configured to host at least one user account and/or at least one client account, and/or configured to operate to transfer data between one or more of these accounts using one or more processors 132.

With the above embodiments in mind, it is understood that the system is configured to implements various computer-implemented program steps involving data stored one or more non-transitory computer media according to some embodiments. In some embodiments, the above-described databases and models described throughout this disclosure are configured to store analytical models and other data on non-transitory computer-readable storage media within the computer system 110 and on computer-readable storage media coupled to the computer system 110 according to some embodiments. In addition, in some embodiments, the above-described applications of the system are stored on computer-readable storage media within the computer system 110 and on computer-readable storage media coupled to the computer system 110. In some embodiments, these operations are those requiring physical manipulation of structures including electrons, electrical charges, transistors, amplifiers, receivers, transmitters, and/or any conventional computer hardware in order to transform an electrical input into a different output. In some embodiments, these structures include one or more of electrical, electromagnetic, magnetic, optical, and/or magneto-optical signals capable of being stored, transferred, combined, compared and otherwise manipulated. In some embodiments, the computer system 110 comprises at least one computer readable medium 136 coupled to at least one of at least one data source 137a, at least one data storage 137b, and/or at least one input/output 137c. In some embodiments, the computer system 110 is embodied as computer readable code on a computer readable medium 136. In some embodiments, the computer readable medium 136 includes any data storage that stores data, which is configured to thereafter be read by a computer (such as computer 140). In some embodiments, the non-transitory computer readable medium 136 includes any physical or material medium that is used to tangibly store the desired information, steps, and/or instructions and which is configured to be accessed by a computer 140 or processor 132. In some embodiments, the non-transitory computer readable medium 136 includes hard drives, network attached storage (NAS), read-only memory, random-access memory, FLASH based memory, CD-ROMs, CD-Rs, CD-RWs, DVDs, magnetic tapes, and/or other optical and non-optical data storage. In some embodiments, various other forms of computer-readable media 136 are configured to transmit or carry instructions to one or more remote computers 140 and/or at least one user 131, including a router, private or public network, or other transmission or channel, both wired and wireless. In some embodiments, the software application modules 138 are configured to send and receive data from a database (e.g., from a computer readable medium 136 including data sources 137a and data storage 137b that comprises a database), and data is configured to be received by the software application modules 138 from at least one other source. In some embodiments, at least one of the software application modules 138 are configured to be implemented by the computer system 110 to output data to at least one user 131 via at least one graphical user interface rendered on at least one digital display.

In some embodiments, the one or more non-transitory computer readable 136 media are distributed over a conventional computer network via the network interface 135a where some embodiments stored the non-transitory computer readable media are stored and executed in a distributed fashion. For example, in some embodiments, one or more components of the computer system 110 are configured to send and/or receive data through a local area network (“LAN”) 139a and/or an internet coupled network 139b (e.g., such as a wireless internet). In some embodiments, the networks 139a, 139b include one or more wide area networks (“WAN”), direct connections (e.g., through a universal serial bus port), or other forms of computer-readable media 136, and/or any combination thereof.

In some embodiments, components of the networks 139a, 139b include any number of personal computers 140 which include for example desktop computers, laptop computers, and/or any fixed, generally non-mobile internet appliances coupled through the LAN 139a. For example, some embodiments include one or more personal computers 140, databases 141, and/or servers 142 coupled through the LAN 139a that are configured for use by any type of user including an administrator. Some embodiments include one or more personal computers 140 coupled through network 139b. In some embodiments, one or more components of the computer system 110 are configured to send or receive data through an internet network (e.g., such as network 139b). For example, some embodiments include at least one user 131a, 131b, coupled wirelessly and accessing one or more software modules of the system including at least one enterprise application 138 via an input and output (“I/O”) 137c. In some embodiments, the computer system 110 is configured to enable at least one user 131a, 131b, to be coupled to access enterprise applications 138 via an I/O 137c through LAN 139a. In some embodiments, the user 131 includes a user 131a coupled to the computer system 110 using a desktop computer, and/or laptop computers, or any fixed, generally non-mobile internet appliances coupled through the internet 139b. In some embodiments, the user includes a mobile user 131b coupled to the computer system 110. In some embodiments, the user 131b connects using any mobile computing 131c to wireless coupled to the computer system 110, including, but not limited to, one or more personal digital assistants, at least one cellular phone, at least one mobile phone, at least one smart phone, at least one pager, at least one digital tablets, and/or at least one fixed or mobile internet appliances.

In some embodiments, the subject matter described herein are directed to technological improvements to the field of NMES by providing electrode pre-positioned on templates that are able to be applied before being covered by a garment and/or being attached to a controller. The disclosure describes the specifics of how a system including a machine with one or more computers comprising one or more processors and one or more non-transitory computer readable media implement the system and its improvements over the prior art. The instructions executed by the machine cannot be performed in the human mind or derived by a human using a pen and paper but require the machine to convert process input data to useful output data. Moreover, the claims presented herein do not attempt to tie-up a judicial exception with known conventional steps implemented by a general-purpose computer; nor do they attempt to tie-up a judicial exception by simply linking it to a technological field. Indeed, the systems and methods described herein were unknown and/or not present in the public domain at the time of filing, and they provide technologic improvements advantages not known in the prior art. Furthermore, the system includes unconventional steps that confine the claim to a useful application.

It is understood that the system is not limited in its application to the details of construction and the arrangement of components set forth in the previous description or illustrated in the drawings. The system and methods disclosed herein fall within the scope of numerous embodiments. The previous discussion is presented to enable a person skilled in the art to make and use embodiments of the system. Any portion of the structures and/or principles included in some embodiments can be applied to any and/or all embodiments: it is understood that features from some embodiments presented herein are combinable with other features according to some other embodiments. Thus, some embodiments of the system are not intended to be limited to what is illustrated but are to be accorded the widest scope consistent with all principles and features disclosed herein.

Some embodiments of the system are presented with specific values and/or setpoints. These values and setpoints are not intended to be limiting and are merely examples of a higher configuration versus a lower configuration and are intended as an aid for those of ordinary skill to make and use the system.

Any text in the drawings are part of the system's disclosure and is understood to be readily incorporable into any description of the metes and bounds of the system. Any functional language in the drawings is a reference to the system being configured to perform the recited function, and structures shown or described in the drawings are to be considered as the system comprising the structures recited therein. Any figure depicting a content for display on a graphical user interface is a disclosure of the system configured to generate the graphical user interface and configured to display the contents of the graphical user interface. It is understood that defining the metes and bounds of the system using a description of images in the drawing does not need a corresponding text description in the written specification to fall with the scope of the disclosure.

Furthermore, acting as Applicant's own lexicographer, Applicant imparts the explicit meaning and/or disavow of claim scope to the following terms:

Applicant defines any use of “and/or” such as, for example, “A and/or B,” or “at least one of A and/or B” to mean element A alone, element B alone, or elements A and B together. In addition, a recitation of “at least one of A, B, and C,” a recitation of “at least one of A, B, or C,” or a recitation of “at least one of A, B, or C or any combination thereof” are each defined to mean element A alone, element B alone, element C alone, or any combination of elements A, B and C, such as AB, AC, BC, or ABC, for example.

“Substantially” and “approximately” when used in conjunction with a value encompass a difference of 5% or less of the same unit and/or scale of that being measured.

“Simultaneously” as used herein includes lag and/or latency times associated with a conventional and/or proprietary computer, such as processors and/or networks described herein attempting to process multiple types of data at the same time. “Simultaneously” also includes the time it takes for digital signals to transfer from one physical location to another, be it over a wireless and/or wired network, and/or within processor circuitry.

As used herein, “can” or “may” or derivations there of (e.g., the system display can show X) are used for descriptive purposes only and is understood to be synonymous and/or interchangeable with “configured to” (e.g., the computer is configured to execute instructions X) when defining the metes and bounds of the system. The phrase “configured to” also denotes the step of configuring a structure or computer to execute a function in some embodiments.

In addition, the term “configured to” means that the limitations recited in the specification and/or the claims must be arranged in such a way to perform the recited function: “configured to” excludes structures in the art that are “capable of” being modified to perform the recited function but the disclosures associated with the art have no explicit teachings to do so. For example, a recitation of a “container configured to receive a fluid from structure X at an upper portion and deliver fluid from a lower portion to structure Y” is limited to systems where structure X, structure Y, and the container are all disclosed as arranged to perform the recited function. The recitation “configured to” excludes elements that may be “capable of” performing the recited function simply by virtue of their construction but associated disclosures (or lack thereof) provide no teachings to make such a modification to meet the functional limitations between all structures recited. Another example is “a computer system configured to or programmed to execute a series of instructions X, Y, and Z.” In this example, the instructions must be present on a non-transitory computer readable medium such that the computer system is “configured to” and/or “programmed to” execute the recited instructions: “configure to” and/or “programmed to” excludes art teaching computer systems with non-transitory computer readable media merely “capable of” having the recited instructions stored thereon but have no teachings of the instructions X, Y, and Z programmed and stored thereon. The recitation “configured to” can also be interpreted as synonymous with operatively connected when used in conjunction with physical structures.

It is understood that the phraseology and terminology used herein is for description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The previous detailed description is to be read with reference to the figures, in which like elements in different figures have like reference numerals. The figures, which are not necessarily to scale, depict some embodiments and are not intended to limit the scope of embodiments of the system.

Any of the operations described herein that form part of the invention are useful machine operations. The invention also relates to a device or an apparatus for performing these operations. All flowcharts presented herein represent computer implemented steps and/or are visual representations of algorithms implemented by the system. The apparatus can be specially constructed for the required purpose, such as a special purpose computer. When defined as a special purpose computer, the computer can also perform other processing, program execution or routines that are not part of the special purpose, while still being capable of operating for the special purpose. Alternatively, the operations can be processed by a general-purpose computer selectively activated or configured by one or more computer programs stored in the computer memory, cache, or obtained over a network. When data is obtained over a network the data can be processed by other computers on the network, e.g. a cloud of computing resources.

The embodiments of the invention can also be defined as a machine that transforms data from one state to another state. The data can represent an article, that can be represented as an electronic signal and electronically manipulate data. The transformed data can, in some cases, be visually depicted on a display, representing the physical object that results from the transformation of data. The transformed data can be saved to storage generally, or in particular formats that enable the construction or depiction of a physical and tangible object. In some embodiments, the manipulation can be performed by a processor. In such an example, the processor thus transforms the data from one thing to another. Still further, some embodiments include methods can be processed by one or more machines or processors that can be connected over a network. Each machine can transform data from one state or thing to another, and can also process data, save data to storage, transmit data over a network, display the result, or communicate the result to another machine. Computer-readable storage media, as used herein, refers to physical or tangible storage (as opposed to signals) and includes without limitation volatile and non-volatile, removable and non-removable storage media implemented in any method or technology for the tangible storage of information such as computer-readable instructions, data structures, program modules or other data.

Although method operations are presented in a specific order according to some embodiments, the execution of those steps do not necessarily occur in the order listed unless explicitly specified. Also, other housekeeping operations can be performed in between operations, operations can be adjusted so that they occur at slightly different times, and/or operations can be distributed in a system which allows the occurrence of the processing operations at various intervals associated with the processing, as long as the processing of the overlay operations are performed in the desired way and result in the desired system output.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein.

Various features and advantages of the invention are set forth in the following claims.

We claim:

1. A system for positioning electrodes on muscle groups comprising:
an electrode sheet,
a flexible circuit, and
two or more electrodes;
wherein the flexible circuit is coupled to the electrode sheet;
wherein the two or more electrodes are coupled to the electrode sheet; and
wherein the two or more electrodes are positioned on the electrode sheet to each activate a different muscle group.

2. The system of claim 1,
wherein the electrode sheet comprises an adhesive.

3. The system of claim 2,
wherein the adhesive is configured to hold the electrode sheet in fixed position on a user.

4. The system of claim 3,
wherein the adhesive is located between the two or more electrodes;
wherein the electrode sheet is configured to not wrap completely around a user limb.

5. The system of claim 1,
wherein the electrode sheet comprises non-conductive portion; and
wherein at least a portion of each of the two or more electrodes on the electrode sheet comprise a conductive material printed on the non-conductive portion.

6. The system of claim 1,
wherein the electrode sheet comprises a controller coupler;
wherein the controller coupler is positioned on an opposite side of the electrode sheet than the two or more electrodes.

7. The system of claim 6,
wherein the controller coupler comprises a raised portion configured to guide controller inputs to controller contacts on the electrode sheet.

8. The system of claim 1,
further including a flexible garment;
wherein the flexible garment is configured to wrap around a user's limb; and
wherein the flexible garment is configured to secure to itself when wrapped around the user's limb.

9. The system of claim 8,
wherein the flexible garment comprises:
a main body portion,
a left extending arm, and
a right extending arm.

10. The system of claim 9,
wherein the electrode sheet comprises a controller coupler;
wherein the controller coupler comprises a raised portion configured to guide controller inputs from a controller to controller contacts on the electrode sheet;
wherein the main body portion comprises a controller attachment portion comprising an aperture; and
wherein the aperture is configured to enable the raised portion to pass through the aperture.

11. The system of claim 10,
wherein the controller is configured to couple to the controller attachment portion; and
wherein the controller is held in position by the raised portion.

12. The system of claim 11,
wherein the controller coupler, the aperture, and the controller attachment portion all comprise substantially a same shape.

13. The system of claim 11,
wherein the controller is configured to deliver electricity to the two or more electrodes.

14. The system of claim 1,
wherein the electrode sheet includes a thigh sheet; and
wherein each thigh sheet includes an electrode placement for two or more muscle groups from muscle groups comprising a Rectus Femoris muscle group, a Vastus Lateralis muscle group, and/or a Vastus Medialis muscle group.

15. The system of claim 1,
wherein each of the two or more electrodes include a conductive material;
wherein the conductive material includes a grid pattern; and
wherein the grid pattern is printed onto the electrode sheet.
